(19) United States
(12) Patent Application Publication
(10) Pub. No.: US 2024/0290454 A1
(43) Pub. Date: Aug. 29, 2024

(54) MOBILE APPLICATION FOR DETERMINING AND RECORDING MEDICATION

(71) Applicant: RECIPROCAL LABS CORPORATION, San Diego, CA (US)

(72) Inventors: Chase Harrison ACTON, San Francisco, CA (US); Nicholas John HIRONS, London (GB); Benjamin Albert THEYE, San Diego, CA (US); Miranda May FOX, San Francisco, CA (US); Connelly Mason DOAN, Las Vegas, NV (US); Ian Pieter SMEENK, San Francisco, CA (US)

(21) Appl. No.: 18/566,010

(22) PCT Filed: Jun. 1, 2022
(86) PCT No.: PCT/US2022/031813

§ 371 Date: Nov. 30, 2023

Related U.S. Application Data
(60) Provisional application No. 63/195,498, filed on Jun. 1, 2021.

(51) Int. Cl. G16H 20/13; G06V 10/70

(52) U.S. Cl. CPC G16H 20/13; G06V 10/70

(57) ABSTRACT

An application for the identification of medication and the recording of a dosage of such medication is disclosed. An image of an unidentified medicine, such as self-administered medication or a medicament device, is captured from an image sensor on a mobile computing device. The type of medicine is determined based on the captured image. An input interface is displayed to accept a dosage input of the determined medicine. The dosage input of the determined medicine is recorded.

### PRIORITY CLAIM

The present disclosure claims priority to and the benefit of U.S. Provisional Patent Ser. No. 63/195,498, filed Jun. 1, 2021. The contents of that application are hereby incorporated by reference in their entirety.

### TECHNICAL FIELD

The present disclosure relates generally to medication tracking, and more specifically to a mobile device that identifies medication from an image and allows recording of a dosage of the identified medication.

### BACKGROUND

Currently, patients are prescribed medications that must be taken at certain times during a day. Further, such patients may need to take multiple types of medications at different times. For example, many patients with respiratory ailments are provided an inhaler to provide dosages of drugs. An asthma patient may be provided a stimulant to assist mucus production and reduce inflammation in clearing up breathing passages. Thus, when the patient experiences asthma exacerbations, the patient can put the inhaler in front their mouth and activate the inhaler spray, delivering a dose of the drug into the lungs in order to relieve the symptom.

It is desirable for health care providers to be able to determine when a patient administers the medication from the inhaler. For example, such data may allow a health care provider to determine whether the medication is effective to treat the respiratory ailment. Such data may also allow a health care provider to determine whether a patient is following recommended treatment. Also, the data may be used to support refills by tracking the doses that remain available.

Currently, a sensor may be attached to certain models of inhalers to collect activation data and transmit that data to a mobile device, such as a smart phone. One example of such a device is the Sensor Model 2016-M, Sensor for MDI manufactured by Propeller Health of Madison, WI. Although such sensors collect useful data, there is no uniform sensor for every type of medication and medication application device. Further, employing a sensor requires correct sequencing and procedure, assuming a sensor is available for the type of prescribed medication. The health provider must select the correct medication application device during enrollment. A sensor must be available for the selected medication application device. If a sensor is available, the patient must log in and set up a matching application on their mobile device. The sensor is typically then delivered to the patient, and the patient must properly attach it to the medication application device. The sensor must be synchronized with the application.

Thus, the most advanced current inhaler systems allow physicians and/or payors to determine that the pump on an inhaled drug has been activated and therefore track the medication. However, this process requires a specialized sensor for each type of medication. Unfortunately, there are numerous inhalers and medication that do not have built-in sensors or are compatible with attachable sensors. Further, other medications in traditional pill forms cannot be tracked by sensors. Another solution for providing tracking of medication may be the provision of an application on a personal mobile device such as a smartphone. When a patient uses an inhaler or takes other medication, the patient may record the dose on the application, and thus provide the activation data. However, such applications are often difficult to use and cannot account for all possible medications, especially when new types of medications become available.

Identification of proper medication for patients that require multiple types of medication is therefore challenging. Patients, caregivers, and care teams are required to change practices or learn new behaviors especially during onboarding of new medications. Reducing the friction that comes with these near-term challenges is critical to ensure the benefits of digital health are realized.

There is a need for a device that may be configured to allow efficient and accessible identification of medications. There is a further need for a device that may be configured to record the dosage taken of the identified medication. There is a further need for a non-medication application device related sensor solution to identifying and recording dosages.

### SUMMARY

One disclosed example is a method of determining medication events. An image of a medicine is captured from an image sensor on a mobile computing device. The type of medicine is determined based on the captured image. An input interface is displayed on a display to accept a dosage input of the determined medicine. A dosage input of the determined medicine is recorded.

A further implementation of the example method is where the medicine is one of an oral administered medication or a medication administered by a medicament device. Another implementation is where the medicament device is an inhaler. Another implementation is where the method includes assigning a time stamp to the recorded dosage input; and transmitting the recorded dosage input, the time stamp and the associated identified medication via a transceiver to a server. Another implementation is where the determining of the type of medicine is performed by a simple machine learning engine trained with a training set of medicine related images. Another implementation is where the simple machine learning engine includes a last layer of a complex machine learning model. Another implementation is where the method further includes storing the determined medicine in a memory device; and generating an interface showing the determined medicine in subsequent recording of dosage inputs of the determined medicine. Another implementation is a computer program product comprising instructions which, when executed by a computer, cause the computer to carry out the methods above. Another implementation is where the computer program product is a non-transitory computer readable medium.

Another disclosed example is a mobile device allowing collection of medication use data. The mobile device includes an image sensor, a memory device, and a display. A processor is coupled to the image sensor, the display, and the memory device. The processor operable to capture an image of a medicine from the image sensor. The processor is operable to determine the type of medicine based on the captured image. The processor is operable to display an input interface on the display to accept a dosage input of the determined medicine. The processor is operable to record a dosage input of the determined medicine in the memory device.

A further implementation of the example mobile device is where the medicine is one of an oral administered medication or a medication administered by a medicament device. Another implementation is where the medicament device is an inhaler. Another implementation is where the mobile device includes a transceiver and where the processor is further operable to: assign a time stamp to the recorded dosage input; and transmit the recorded dosage input, the time stamp and the associated identified medication via the transceiver to a server. Another implementation is where the processor is operable to execute a simple machine learning engine trained with a training set of medicine related images to determine the medicine. Another implementation is where the simple machine learning engine includes a last layer of a complex machine learning model. Another implementation is where the processor is further operable to store the determined medicine in the memory device; and generate an interface showing the determined medicine in subsequent recording of dosage inputs of the determined medicine.

Another disclosed example is a data collection system. The system includes a mobile computing device having an image sensor operable to capture an image of a medicine, a storage device, a transceiver, a display, and a processor. The processor is operable to classify the medicine; display an input for dosage of the classified medicine on the display; and record an input of the dosage of the classified medicine on the storage device. The processor is operable to send the collected dosage and classified medicine data via the transceiver. The system includes an analysis server receiving the collected data and recording the dosage and the classified medicine data in relation to a patient.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1 is a diagram of a medication identification and use data collection system;

FIG. 2 is a diagram of a computing device in FIG. 1;

FIGS. 3A-3D are screen shots of interfaces generated by the example application that identifies and records medications;

FIG. 4 is a block diagram of the machine learning model that identifies medication from a scanned image; and

FIG. 5 is a flow diagram of the routine for a mobile device for identifying and recording doses of medication.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

The present disclosure relates to an application that may facilitate recognition of medication without a specialized sensor for a medication application device such as an inhaler. The application leverages an image sensor, such as a camera, on a personal mobile device to capture an image of a medication or a medicament device. The application classifies and identifies the medication or the medicament device based on the image. Other information may be gathered in addition from performing OCR on the label to assist in identifying the medication and confirming the image analysis. After the medication is identified, the patient may activate a pop-up window to record the dosage of the medication or the dosage applied by the medicament device. In this manner, accurate recording of medication may be stored for the patient. Such data may be sent to a remote server for update of a medical record of the patient.

A transfer learning routine reduces the need for gathering large amounts of medication data to train a medication identification machine learning model for the identification of the medication or the medicament device. In this example the core machine learning framework allows for out of the box image classification of images of medication and medicament device. A pre-trained complex computer vision model has a last layer with the most meaningful information. The last layer of the pre-trained complex computer vision model is transferred to the new task of identification of medication and medicament devices. A simple machine learning model is applied on the last layer allowing medication and medicament labels to be classified accurately without a large data set.

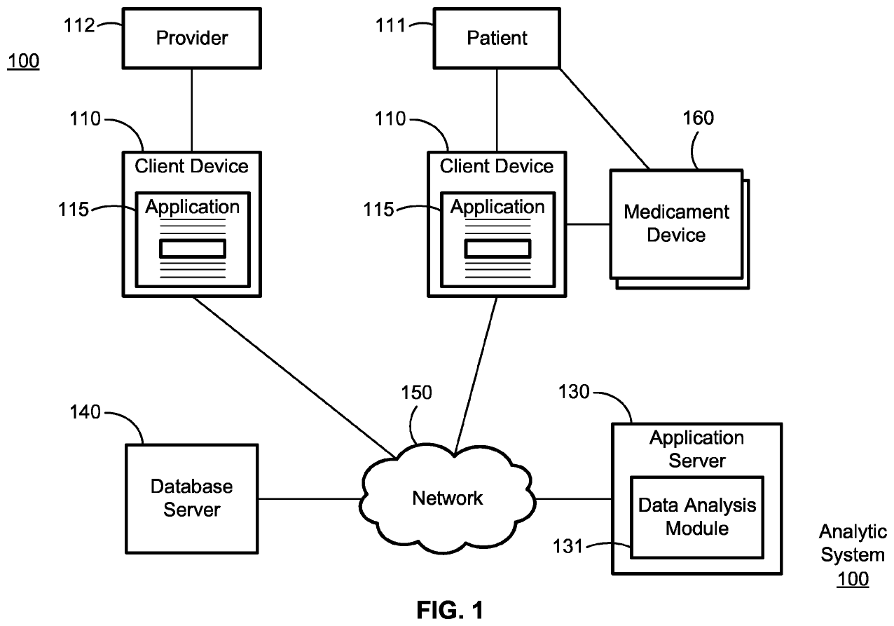

FIG. 1 shows a medication identification and dosage analytics system 100 for collecting data based on identifying medication and medicament devices, monitoring accurate, real-time medicament device and medication events, and performing analytics on that data for a general patient population, according to one embodiment.

The analytics system 100 includes client computing devices 110, a medicament device 160, an application server 130, a database server 140, and a network 150. Although FIG. 1 illustrates only a single instance of most of the components of the analytics system 100, in practice more than one of each component may be present, and additional or fewer components may be used. In particular, there may be multiple medicament devices 160 as well as other medications 162 in an orally administered format such as a capsule or tablet for a single patient 111. Both the medications 162 and the medicament devices 160 are generally referenced as medicine that the patient must take according to a treatment plan. Other types of medications may be determined using the techniques described herein.

The client devices 110, at the behest of their users, interact with the analytics system 100 via the network 150. In this example, the medications 162 and the medications administered through the medicament devices 160 are geared toward a respiratory ailment, but other types of medication directed toward other ailments may use the principles described herein. For purposes of explanation and clarity it is useful to identify at least two different types of users in the field of respiratory ailments. In this example, the patient 111 is a user burdened with a respiratory ailment such as asthma or COPD who makes use of the respiratory ailment analytics system 100 at least in part to obtain personalized rescue event risk notifications provided by the server 130 and management notifications created by their health care provider 112 in this example. Such notifications can be provided in exchange for the user's permission to allow the respiratory ailment analytics system 100 to monitor the patient's 111 medicament device 160 usage or usage of the medications 162. As will be explained below, medication events such as the patient 111 activating the medicament device 160 or taking medications 162 are recorded through an application 115 executed by the client device 110. In this example, the application 115 activates an image sensor such as a camera on the client device 110 associated with the patient. The medicament device 160 or medication 162 may be identified by a captured image of the medicament device 160 or the medication 162. The application 115 allows a patient to indicate the use of the medicament device 160 or taking the medication 162, and the application 115 reports the event to the application server 130. In this example, the patients 111 represent a patient population, for which individual data is taken for each patient in the group. Another type of user may use the medicament device 160 for control medication to control symptoms of respiratory ailment. Other users may use different devices or different medications to treat or prevent other types of ailments.

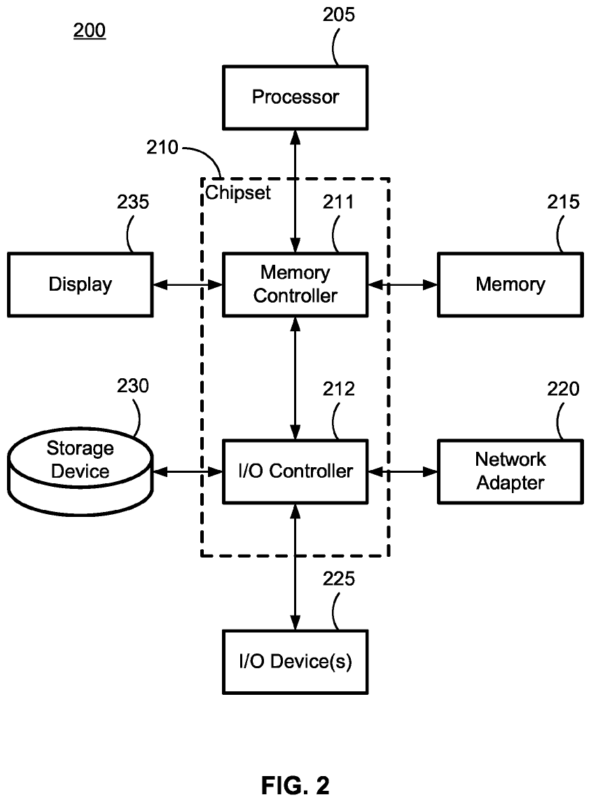

The client device 110 is a computer system. An example physical implementation is described more completely below with respect to FIG. 2. The client device 110 is configured to wirelessly communicate with the respiratory ailment analytics system 100 via network 150. With network 150 access, the client device 110 transmits to system 100 the user's geographical location and the time of a rescue or control medication event, as well as information describing the event as received from the application on the client device 110.

Regarding user location and event times, the client device 110 may determine the geographical location and time of a rescue or control event through use of information about the cellular or wireless network 150 to which it is connected. For example, the current geographical location of the client device 110 may be determined by directly querying the software stack providing the network 150 connection. Alternatively, the geographical location information may be obtained by pinging an external web service (not shown in FIG. 1) made accessible via network 150. The time of an event can be provided by the application 115 as part of the event data or added to event data by querying an appropriate software routine available as part of the client device's native operating system.

The application 115 provides a user interface (herein referred to as a “dashboard”) that is displayed on a screen of the client device 110 and allows a user to input commands to control the operation of the application 115. The dashboard is the mechanism by which healthcare providers 112 and patients 111 access the resources managed by the system 100. For example, the dashboard allows patients 111 and providers 112 to interact with each other, receive asthma rescue event risk notifications, exchange messages about treatment, provide and receive additional event and non-event data, and so on. The application 115 may be coded as a web page, series of web pages, or content otherwise coded to render within an internet browser. Application 115 may also be coded as a proprietary application configured to operate on the native operating system of the client device 110.

In addition to providing the dashboard, application 115 may also perform some data processing on rescue or control event data locally using the resources of client device 110 before sending the processed data through the network 150. Thus, in this example the application 115 allows communication of event data in relation to the operation of the medicament device 160. Event data sent through the network 150 is received by the application server 130 where it is analyzed and processed for storage and retrieval in conjunction with database server 140. The application server 130 may direct retrieval and storage request to the database server 140 as required by the client application 115. As will be explained, this analysis may be extended to include event data from multiple patients 111.

The example medicament device 160 is a medical device used to deliver medication to the lungs of a user experiencing a respiratory ailment rescue event. Different medicaments are used for asthma and COPD. Different medicaments may also be used for rescue and control for asthma and COPD. Medicament devices (e.g., inhalers) are typically portable and small enough to be carried by hand for ease of accessibility when treating respiratory symptoms. In one embodiment, medicine is delivered in aerosol form through a medicament device 160 such as a metered dose inhaler. Metered dose inhalers included a pressured, propellant canister of aerosol medicine, a metering valve for delivering a regulated medicine dosage amount, and a plastic holder that holds the pressurized canister and also forms a mouthpiece for delivery of the medicine. In another embodiment, medicine is delivered in dry powder form through a medicament device 160 such as a dry powder inhaler. Dry powder inhalers may have Cartesian ovular shaped bodies that house wheel and gear mechanisms enabling a user to index through a strip of dry powder medication. The bodies of dry powder inhalers also include a manifold and a mouthpiece to deliver dry powder to the user. Although a medicament device is used in this example, it is understood the churn analysis may be used for any personal treatment device that requires the user to use the device on a periodic basis. Further, although the above examples relate to respiratory disorders, the principles described herein may be applied to treatment regimens for other types of ailments. Other devices such as auto-injectors and nebulizers may also deliver medication and utilize the principles herein.

The medications 162 may be in any form that may be self-administered by the patient 111 without the assistance of a device such as an inhaler. Thus, the medications 162 may include tablets, gels, lozenges and patches; nasal spray; suppositories, and the like.

Each patient may be associated with more than one medicament device 160 or medications 162. For example, the patient may have a rescue medicament device 160 that dispenses rescue medication, and a controller medicament device 160 that dispenses controller medication. Other medications 162 may be in the form of different shaped and colored orally ingested tablets or capsules.

As introduced above, the application 115 generates an interface to capture data about usage of the medicament device 160 or medication 162. Specifically, the application 115 captures the time and geographical location of the rescue medication event or controller medication event, that is, usages of the rescue or controller medicament device 160, by the patient 111. The application 115 also captures a patient taking doses of other medication 162. Each device 110 transmits the collected event data in real-time or as soon as a network connection is achieved, automatically without input from the patient 111 or health care provider 112. The medication event information is sent to the application server 130 for use in analysis, generation of asthma rescue event notifications, and in aggregate analyses of event data across multiple patients for the purpose of determining churn.

The application server 130 is a computer or network of computers. Although a simplified example is illustrated in FIG. 2, typically the application server will be a server class system that uses powerful processors, large memory, and faster network components compared to a typical computing system used, for example, as a client device 110. The server typically has large secondary storage, for example, using a RAID (redundant array of independent disks) array and/or by establishing a relationship with an independent content delivery network (CDN) contracted to store, exchange and transmit data such as the asthma notifications contemplated above. Additionally, the computing system includes an operating system, for example, a UNIX operating system, LINUX operating system, or a WINDOWS operating system. The operating system manages the hardware and software resources of the application server 130 and also provides various services, for example, process management, input/output of data, management of peripheral devices, and so on. The operating system provides various functions for managing files stored on a device, for example, creating a new file, moving or copying files, transferring files to a remote system, and so on.

The application server 130 includes a software architecture for supporting access and use of the analytics system 100 by many different client devices 110 through network 150, and thus at a high level can be generally characterized as a cloud-based system. The application server 130 generally provides a platform for patients 111 and healthcare providers 112 to report data recorded by the application identifying and recording use of their medicament devices 160 including both rescue medication and controller medication events, collaborate on respiratory ailment treatment plans, browse and obtain information relating to their condition and geographic location, and make use of a variety of other functions. The application server 130 also is a platform for tracking the events for other types of medicament devices and medications 162 and perform similar functions such as collaborating on treatment plans, browse and obtaining information relating to their condition and geographic location, and making use of a variety of other functions.

Generally, the application server 130 is designed to handle a wide variety of data. The application server 130 includes logical routines that perform a variety of functions including checking the validity of the incoming data, parsing and formatting the data if necessary, passing the processed data to a database server 140 for storage, and confirming that the database server 140 has been updated.

The application server 130 stores and manages data at least in part on a patient by patient basis. Towards this end, the application server 130 creates a patient profile for each user. The patient profile is a set of data that characterizes the patient 111 of the respiration ailment analytics system 100. The patient profile may include identity information about the patient such as age, gender, current rescue medication, current controller medication, notification preferences, a controller medication adherence plan, a relevant medical history, and a list of non-patient users authorized to access to the patient profile. The profile may further specify a device identifier, such as a unique media access control (MAC) address identifying the one or more client devices 110 or sensors 120 authorized to submit data (such as controller and rescue medication events) for the patient.

The profile may specify which different types of notifications are provided to patients 111 and their personal healthcare providers 112, as well as the frequency with which notifications are provided. For example, a patient 111 may authorize a healthcare provider 112 to receive notifications indicating a rescue event. The patient 111 may also authorize their healthcare provider 112 be given access to their patient profile and rescue event history. If the healthcare provider 112 is provided access to the patient profile of the patient 111, the healthcare provider may specify controller adherence or rescue medication plans for the corresponding COPD or asthma condition. Medication plans may include a prescribed number of doses per day for controller medications.

The application server 130 also creates profiles for health care providers 112. A health care provider profile may include identifying information about the health care provider 112, such as the office location, qualifications and certifications, and so on. The health care provider profile also includes information about their patient population. The provider profile may include access to all of the profiles of that provider's patients, as well as derived data from those profiles such as aggregate demographic information, rescue and controller medication event patterns, and so on. This data may be further subdivided according to any type of data stored in the patient profiles, such as by geographic area (e.g., neighborhood, city) over by time period (e.g., weekly, monthly, or yearly).

For example, the application server 130 receives rescue medication event information from the client device 110, triggering a variety of routines on the application server 130. In the example implementations described below, the data analysis module 131 executes routines to access event data, sync data, as well as other data including a patient's profile, analyze the data, and output the results of its analysis to both patients 111 and providers 112.

Other types of analyses may include daily/weekly adherence trends, adherence changes over time, adherence comparisons to other relevant populations (e.g., all patients, patients on a particular rescue medication or controller medication or combination thereof, identification of triggers (spatial, temporal, environmental), rescue use trends over time, and rescue use comparisons to other relevant populations.

Responsive to any analyses performed, the application server 130 prepares and delivers push notifications to send to patients 111, authorized healthcare providers 112, and/or other users provided access to the patient's profile. Notifications can provide details about the timing, location, and affected patient(s) 111 involved in a medication rescue event. Notifications may additionally comprise a distress or emergency signal that requests emergency assistance that are distributed to emergency assistance providers 112. Notifications may also include the results of the churn analysis performed by the data analysis module 131. More information regarding the types of notifications that may be sent and the content they may contain is further described below.

In addition to providing push notifications in response to an asthma or COPD exacerbation risk analysis, notifications may also be provided as pull notifications, at particular time intervals. Additionally, some notifications (whether push or pull) may be triggered not in response to an asthma or COPD exacerbation risk analysis performed in response to a rescue medication event, but instead in response to a risk analysis performed in response to one of the underlying factors in the asthma or COPD exacerbation risk analysis changing, such that an updated notification is warranted. For example, if weather conditions indicate that an increase in air pollution is occurring or is imminent, this may trigger the carrying out of asthma exacerbation risk analyses for all patients located in the particular geographic area where the pollution is occurring.

Notifications are provided through the network 150 to client applications 115 in a data format specifically designed for use with the client applications, and additionally or alternatively may be provided as short message service (SMS) messages, emails, phone calls, or in other data formats communicated using other communication mediums.

The database server 140 stores patient and provider data related data such as profiles, medication events, patient medical history (e.g., electronic medical records). Patient and provider data are encrypted for security and is at least password protected and otherwise secured to meet all Health Insurance Portability and Accountability Act (HIPAA) requirements. Any analyses (e.g., asthma risk analyses) that incorporate data from multiple patients (e.g., aggregate rescue medication event data) and are provided to users is de-identified so that personally identifying information is removed to protect patient privacy. The database server 140 may also include customer service information in relation to the patient 111 from the health care providers 112 such as inquiries, requests for assistance and the like.

The database server 140 may also store non-patient data. This data includes regional data about a number of geographic regions such as public spaces in residential or commercial zones where patients are physically located and may be exposed to pollutants. This data may specifically include or be processed to obtain a patient's proximity to green space (areas including concentrated numbers of trees and plants). One example of regional data includes georeferenced weather data, such as temperature, wind patterns, humidity, the air quality index, and so on. Another example is georeferenced pollution data, including particulate counts for various pollutants at an instance of time or measured empirically. The regional data includes information about the current weather conditions for the time and place of the rescue event such as temperature, humidity, air quality index. All of the items of data above may vary over time, and as such the data itself may be indexed by time, for example separate data points may be available by time of day (including by minute or hour), or over longer periods such as by day, week, month, or season. Although the database server 140 is illustrated in FIG. 1 as being an entity separate from the application server 130 the database server 140 may alternatively be a hardware component that is part of another server such as server 130, such that the database server 140 is implemented as one or more persistent storage devices, with the software application layer for interfacing with the stored data in the database is a part of that other server 130.

The collected usage data for medication may be used by the application server 130 for different applications. For example, the event data may be used to monitor the patient taking certain medication in accordance with compliance rules that specify the required medication usage over a compliance period, such as 30 days, in terms of a minimum number of doses, such as four times, for some minimum number of days, e.g., 21 days, within the compliance period. The summary data post-processing may determine whether the most recent time period is a compliant session by comparing the usage time with the minimum duration from the compliance rule. The results of such post-processing are referred to as “compliance data.” Such compliance data may be used by a health care provider 112 to tailor therapy that may include the inhaler and other mechanisms. Other actors such as payors may use the compliance data to determine whether reimbursement may be made to a patient. The application server 130 may execute applications that have other health care functions such as determining overall use of medication based on collection of data from numerous patients. For example, health care organizations may run analysis algorithms to determine correlations between populations of patients using their drugs effectively and reduced hospitalization rates. Another example is determination of physiological patterns of pre- and post-drug use that indicate if a patient is at risk of imminent rehospitalization. Another example is an analysis of the effectiveness of different drug brands (but same drug type) in managing patient health.

Another example, may be customization of treatment to a patient. The dosage application and the corresponding data such as triggers and symptoms from other sensors and databases may be used to determine a customized dosage for the individual user. Other stored data may be used to further tailor a dosage. The customized dosage may be therefore determined based on the physiology, disease type and activity specific to the user. The additional data may be stored in the database 140 and be used by the application server 130 to assist in personalized analysis of the data received from mobile device 110. Other analysis may be performed. For example, if a pattern is identified with pollen or other environmental triggers from the collected data, then external weather data could be used to notify the patient of the likelihood of a needed dosage. A pattern of a particular activity followed by a medication usage may trigger intervention if the pattern is broken. Thus, if the patient always takes a dosage after they go for a run every Tuesday, and they log a run Tuesday but no medication usage, then the system may send a notification to the patient reminding them. If patient-reported sleep quality is steadily declining based on the inputs collected by a universal sensor, an intervention by a physician can be triggered. If the user fails to perform a specific scheduled activity (such as exercise) for a period as determined from the collected data, the analysis may determine that the patient is subject to an impending exacerbation or a general decline of well-being.

The database server 140 stores data according to defined database schemas. Typically, data storage schemas across different data sources vary significantly even when storing the same type of data including cloud application event logs and log metrics, due to implementation differences in the underlying database structure. The database server 140 may also store different types of data such as structured data, unstructured data, or semi-structured data. Data in the database server 140 may be associated with patients, groups of patients, and/or entities. The database server 140 provides support for database queries in a query language (e.g., SQL for relational databases, JSON NoSQL databases, etc.) for specifying instructions to manage database objects represented by the database server 140, read information from the database server 140, or write to the database server 140.

The network 150 represents the various wired and wireless communication pathways between the client 110 devices, the application server 130, and the database server 140. Network 150 uses standard Internet communications technologies and/or protocols. Thus, the network 150 can include links using technologies such as Ethernet, IEEE 802.11, integrated services digital network (ISDN), asynchronous transfer mode (ATM), etc. Similarly, the networking protocols used on the network 150 can include the transmission control protocol/Internet protocol (TCP/IP), the hypertext transport protocol (HTTP), the simple mail transfer protocol (SMTP), the file transfer protocol (FTP), etc. The data exchanged over the network 150 can be represented using technologies and/or formats including the hypertext markup language (HTML), the extensible markup language (XML), etc. In addition, all or some links can be encrypted using conventional encryption technologies such as the secure sockets layer (SSL), Secure HTTP (HTTPS) and/or virtual private networks (VPNs). In another embodiment, the entities can use custom and/or dedicated data communications technologies instead of, or in addition to, the ones described above.

FIG. 2 is a high-level block diagram illustrating physical components of an example computer 200 that may be used as part of a client device 110, application server 130, and/or database server 140 from FIG. 1, according to one embodiment. Illustrated is a chipset 210 coupled to at least one processor 205. Coupled to the chipset 210 is volatile memory 215, a network adapter 220, an input/output (I/O) device(s) 225, a storage device 230 representing a non-volatile memory, and a display 235. In one embodiment, the functionality of the chipset 210 is provided by a memory controller 211 and an I/O controller 212. In another embodiment, the memory 215 is coupled directly to the processor 205 instead of the chipset 210. In some embodiments, memory 215 includes high-speed random access memory (RAM), such as DRAM, SRAM, DDR RAM or other random access solid state memory devices.

The storage device 230 is any non-transitory computer-readable storage medium, such as a hard drive, compact disk read-only memory (CD-ROM), DVD, or a solid-state memory device. The memory 215 holds instructions and data used by the processor 205. The I/O device 225 may be a touch input surface (capacitive or otherwise), a mouse, track ball, or other type of pointing device, a keyboard, or another form of input device. The display 235 displays images and other information from the computer 200. The network adapter 220 couples the computer 200 to the network 150.

As is known in the art, a computer 200 can have different and/or other components than those shown in FIG. 2. In addition, the computer 200 can lack certain illustrated components. In one embodiment, a computer 200 acting as server 140 may lack a dedicated I/O device 225, and/or display 218. Moreover, the storage device 230 can be local and/or remote from the computer 200 (such as embodied within a storage area network (SAN)), and, in one embodiment, the storage device 230 is not a CD-ROM device or a DVD device.

Generally, the exact physical components used in a client device 110 will vary in size, power requirements, and performance from those used in the application server 130 and the database server 140. For example, client devices 110, which will often be home computers, tablet computers, laptop computers, or smart phones, will include relatively small storage capacities and processing power, but will include input devices and displays. These components are suitable for user input of data and receipt, display, and interaction with notifications provided by the application server 130. In contrast, the application server 130 may include many physically separate, locally networked computers each having a significant amount of processing power for carrying out the churn risk analyses introduced above. In one embodiment, the processing power of the application server 130 provided by a service such as Amazon Web Services™. Also, in contrast, the database server 140 may include many, physically separate computers each having a significant amount of persistent storage capacity for storing the data associated with the application server.

As is known in the art, the computer 200 is adapted to execute computer program modules for providing functionality described herein. A module can be implemented in hardware, firmware, and/or software. In one embodiment, program modules are stored on the storage device 230, loaded into the memory 215, and executed by the processor 205.

Upon receiving and storing the rescue usage event data, the application server 130 may request further information from the patient describing the rescue or control usage event. To obtain the information, the application server 130 generates a push notification, including the questions to be asked, to be sent to the patient's client device 110. The patient may respond to the request by providing inputs in response to the application. Alternatively, the patient 111 may elect not to respond to the request. This is permissible, any gaps in information may be obtained through later push notifications, or upon entry by provider 112 after a meeting with the patient 111. In one implementation, the failure to receive the additionally requested data in response to request does not hold up the remainder of the analysis.

In addition to requesting additional event data, the application server 130 accesses stored contextual data from the database server 140. Generally, contextual data refers data other than event data, which includes but is not limited to: to atmospheric conditions, weather conditions, air quality conditions, pollen data, patient data recorded from past rescue usage events, and any other considerations that are not directly detected by the medicament device at the time of the rescue usage event. By contrast, event data refers to any parameters related to the rescue event and reported by the medicament device, such as medication dosage, the time of the event, the location of the event, and relevant adherence data. Both forms of data may include temporally-current information as well as stored historical data. Accordingly, as part of obtaining the contextual data, the application server 130 also accesses historical rescue usage event data for the patient 111. This historical data can include all of the data from any past controller or rescue medication event data from the patient's history over a variety of windows of time in the past, and each historical event may include all of the same items of information as was reported for this event along with any data collected upon follow up thereafter.

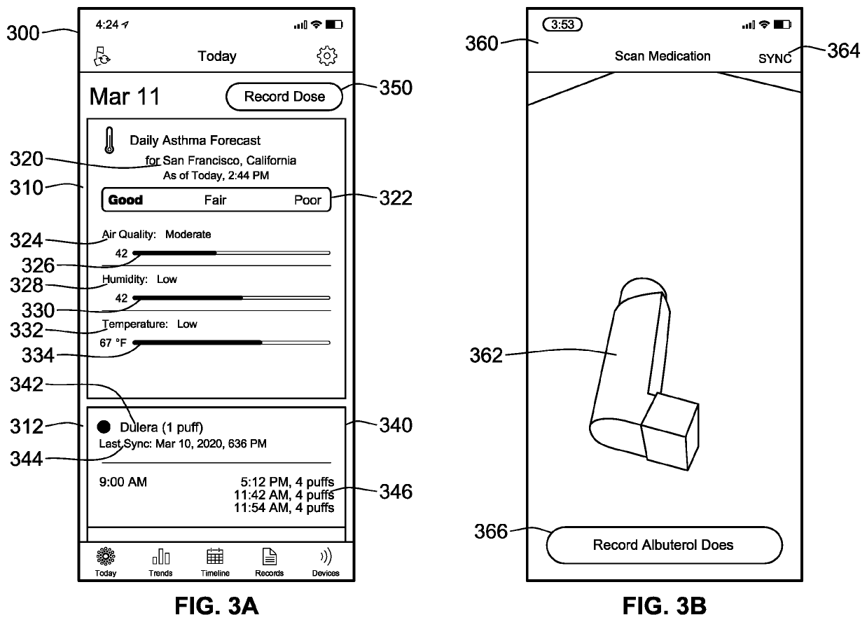

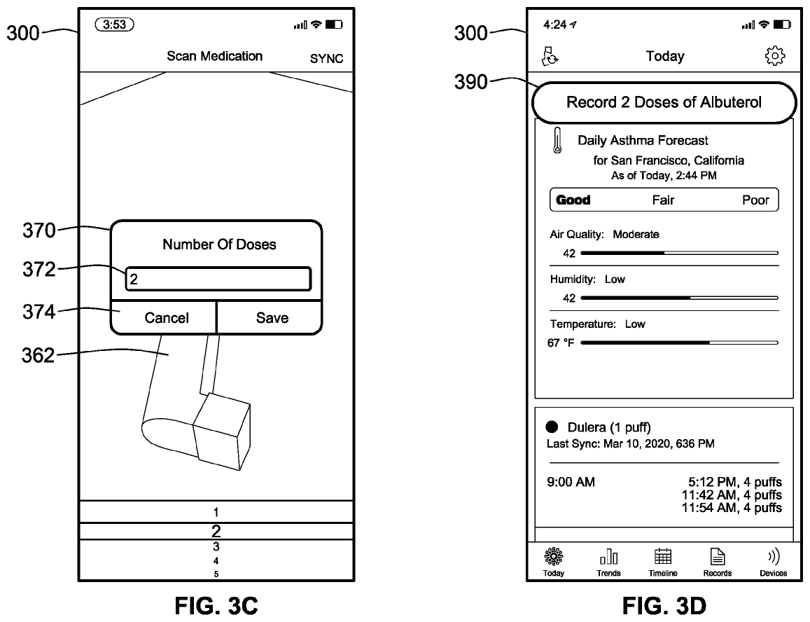

FIG. 3A shows an interface 300 generated by the application 115 in FIG. 1 to capture an image of medication of a medicament device. The interface 300 includes an environment area 310 and a medication summary area 312. In this example, the environment area 310 includes useful environmental information for a patient in relation to potential ailments such as asthma. In this example, the environmental area includes an asthma forecast field 320 and a forecast bar 322 that shows the overall condition in relation to asthma ranging from good to poor. An air quality field 324 displays a score graph 326 for a score of air quality. A humidity field 328 displays a score graph 330 for a percentage of the current humidity. A temperature field 332 displays the current temperature on a temperature graph 334.

The medication summary area 312 lists the medications that the patient has recorded via imaging. Alternatively, known medication images and information may be stored by the application 115. An example medication field 340 includes a description and dosage of a medication 342, information on the last synch 344, and a time summary 346 of the time and puffs of the medication.

In cases where the medication has not been previously identified and therefore is not listed in the medication summary area 312, a record dose button 350 is displayed. Selecting the record dose button 350 results in the generation of a medication capture interface 360 shown in FIG. 3B. The medication capture interface 360 accesses the camera of the mobile device and displays an image 362 of the medication or the medicament device. A save button 364 captures the image 362 of the medication. The application 115 identifies the medication based on the image. On identification of the medication from the image of the medication or the medicament device, a record dose button 366 is displayed with the name or other identification of the medication.

When the record button 366 is selected, a pop-up window 370 is displayed on the interface 360 as shown in FIG. 3C. The pop-up window 370 includes a doses field 372, a cancel button 374 and a save button 376. The doses field 372 allows a user to enter the number of doses the user administers via a selection interface 380. The cancel button 374 cancels the entry of number of doses. When a number is selected, the number of doses may be recorded by selecting the save button 376.

When the save button 376 is selected, the main interface 300 is displayed with a summary bubble 390 interposed as shown in FIG. 3D. The summary bubble 390 shows that the doses of the medication have been recorded. The mobile device 110 may transmit the dosage data, time, location, and other data to the server 130 in FIG. 1.

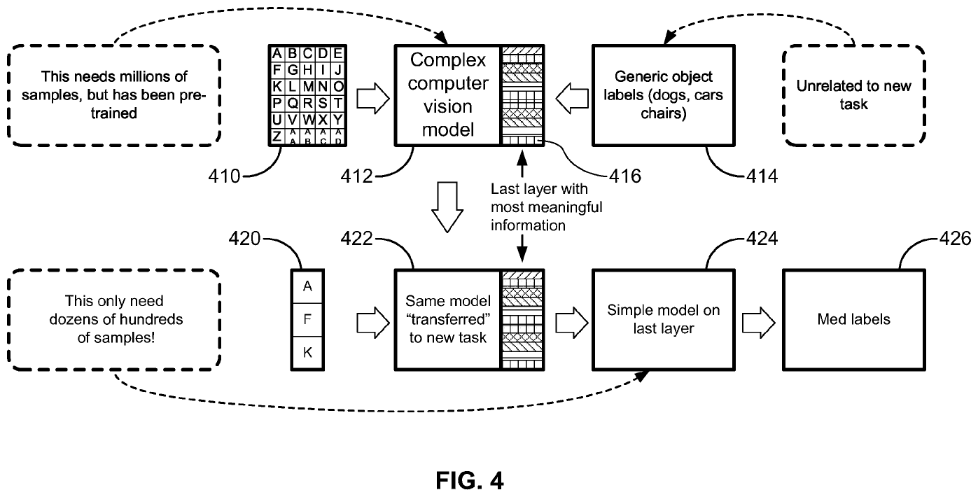

FIG. 4 is a flow diagram of a simplified machine learning system allowing classification of medication on the computing device 110 operated by the patient 111. The classification of medication is a simplified process that takes a more complex layer to allow learning of classification of a discrete class of objects (medication shapes, sizes, and other image characteristics) related to the type of medication.

In this example, a very large image data set 410 is provided to train a complex computer vision model 412. In this example, the complex computer vision model is the Apple Core ML framework for deep learning but any similar framework for image classification may be used. The trained vision model may provide generic object labels 414 to a wide variety of different objects. The vision model 412 includes a last layer 416 with the most meaningful information.

The last layer 416 is incorporated into a simplified model that is transferred to the task of identifying medication images. In this example, a much smaller training set 420 of images of medications and medications in medication dispensers and their respective medications are provided. A transferred model 422 is based on the last layer 416 to produce a simple model 424 only on the last layer 416. The trained simple model 424 may provide an output of labelling medication images 426. The simple model 424 may be saved to the mobile device 110 and accessed by the application 115 during the identification process shown in FIG. 3B. The smaller footprint of the simple model 424 thus allows for the identification process to be performed by the mobile device 110 independently of other networked devices.

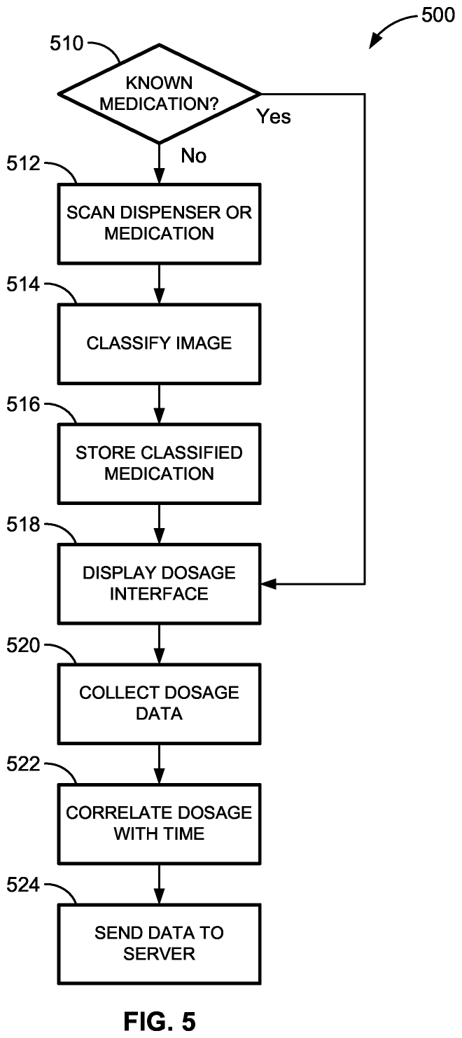

FIG. 5 is a flow diagram 500 of the operational routine executed by the controller the application on the remote mobile device 110 to capture an image of medication and provide a correct interface to record a dosage of the medication. The flow diagram 500 in FIG. 5 is representative of example machine readable instructions for the mobile device 110 in FIG. 1. In this example, the machine readable instructions comprise an algorithm for execution by: (a) a processor; (b) a controller; and/or (c) one or more other suitable processing device(s). The algorithm may be embodied in software stored on tangible media such as flash memory, CD-ROM, floppy disk, hard drive, digital video (versatile) disk (DVD), or other memory devices. However, persons of ordinary skill in the art will readily appreciate that the entire algorithm and/or parts thereof can alternatively be executed by a device other than a processor and/or embodied in firmware or dedicated hardware in a well-known manner (e.g., it may be implemented by an application specific integrated circuit [ASIC], a programmable logic device [PLD], a field programmable logic device [FPLD], a field programmable gate array [FPGA], discrete logic, etc.). For example, any or all of the components of the interfaces can be implemented by software, hardware, and/or firmware. Also, some or all of the machine readable instructions represented by the flowcharts may be implemented manually. Further, although the example algorithm is described with reference to the flowchart illustrated in FIG. 5, persons of ordinary skill in the art will readily appreciate that many other methods of implementing the example machine readable instructions may alternatively be used. For example, the order of execution of the blocks may be changed, and/or some of the blocks described may be changed, eliminated, or combined.

The routine first determines whether an input for known medication has been selected by the user (510). If the medication is not known, the routine displays the interface 360 in FIG. 3B and scans an image of the medicament device or the medication (512). The routine then classifies the unknown medication based on the scanned image (514). The classified medication is then stored in the memory 230 for future reference (516). After the classified medication is stored (516), or if the medication is already known (510), the dosage pop-up window shown in FIG. 3C is displayed along with the type of medication (518).

The user inputs the number of doses and the number of doses is collected by the routine (520). The dosages are correlated with the time and optionally any other relevant background or operational data (522). The mobile device 110 may then transmit the dosage data to the server 130 for further analysis (524). Alternatively, the mobile device 110 may transmit the received data at a predefined period of time. In this example, the predefined period of time may be 24 hours. Alternatively, the server 130 may request transmission of a data packet from the mobile device 110 on a periodic basis or when the server 130 determines a medication or other condition is probably present with the patient based on past patterns.

As used in this application, the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

One or more elements or aspects or steps, or any portion(s) thereof, from one or more of any of claims 1 to 17 below can be combined with one or more elements or aspects or steps, or any portion(s) thereof, from one or more of any of the other claims 1 to 17 or combinations thereof, to form one or more additional implementations and/or claims of the present disclosure.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above described embodiments. Rather, the scope of the invention should be defined in accordance with the following claims and their equivalents.

1. A method of determining medication events, the method comprising:
capturing an image of a medicine from an image sensor on a mobile computing device;
determining the type of medicine based on the captured image;
displaying an input interface on a display to accept a dosage input of the determined medicine; and
recording a dosage input of the determined medicine.

2. The method of claim 1, wherein the medicine is one of an oral administered medication or a medication administered by a medicament device.

3. The method of claim 2, wherein the medicament device is an inhaler.

4. The method of claim 1, further comprising:
assigning a time stamp to the recorded dosage input; and
transmitting the recorded dosage input, the time stamp and the associated identified medication via a transceiver to a server.

5. The method of claim 1, wherein the determining of the type of medicine is performed by a simple machine learning engine trained with a training set of medicine related images.

6. The method of claim 5, wherein the simple machine learning engine includes a last layer of a complex machine learning model.

7. The method of claim 1, further comprising:
storing the determined medicine in a memory device; and
generating an interface showing the determined medicine in subsequent recording of dosage inputs of the determined medicine.

8. A computer program product of claim 1, comprising instructions which, when executed by a computer, cause the computer to carry out the method of.

9. The computer program product of claim 8, wherein the computer program product is a non-transitory computer readable medium.

10. A mobile device allowing collection of medication use data, the mobile device comprising:
an image sensor;
a memory device;
a display; and
a processor coupled to the image sensor, the display, and the memory device, the processor operable to:
capture an image of a medicine from the image sensor;
determine the type of medicine based on the captured image;
display an input interface on the display to accept a dosage input of the determined medicine; and
record a dosage input of the determined medicine in the memory device.

11. The mobile device of claim 10, wherein the medicine is one of an oral administered medication or a medication administered by a medicament device.

12. The mobile device of claim 11, wherein the medicament device is an inhaler.

13. The mobile device of claim 10, further comprising a transceiver, wherein the processor is further operable to:
assign a time stamp to the recorded dosage input; and
transmit the recorded dosage input, the time stamp and the associated identified medication via the transceiver to a server.

14. The mobile device of claim 10, wherein the processor is operable to execute a simple machine learning engine trained with a training set of medicine related images to determine the medicine.

15. The mobile device of claim 12, wherein the simple machine learning engine includes a last layer of a complex machine learning model.

16. The mobile device of claim 10, wherein the processor is further operable to store the determined medicine in the memory device; and generate an interface showing the determined medicine in subsequent recording of dosage inputs of the determined medicine.

17. A data collection system, the system comprising:
a mobile computing device having an image sensor operable to capture an image of a medicine, a storage device, a transceiver, a display and a processor operable to:
classify the medicine;
display an input for dosage of the classified medicine on the display;
record an input of the dosage of the classified medicine;
send the collected dosage and classified medicine data via the transceiver; and
an analysis server receiving the collected data and recording the dosage and the classified medicine data in relation to a patient.
